(19) United States
(12) Patent Application Publication
(10) Pub. No.: US 2017/0112435 A1
(43) Pub. Date: Apr. 27, 2017

(54) Physical Therapy Assembly

(71) Applicant: Julian Samodulski, Glendale, NY (US)

(72) Inventor: Julian Samodulski, Glendale, NY (US)

(21) Appl. No.: 14/921,202

(22) Filed: Oct. 23, 2015

(51) Int. Cl. A61B 5/00; A61B 5/11; A61B 5/107

(52) U.S. Cl. CPC A61B 5/4848; A61B 5/1071; A61B 5/11

(57) ABSTRACT

A physical therapy assembly includes a measuring device that may be manipulated thereby facilitating the measuring device to measure a plurality of range of motion tests. The measuring device comprises a plurality of rulers is movably coupled together. Thus, the measuring device may form at least one selected angle. A plurality of goniometers is provided. Each of the goniometers is rotatably coupled to the measuring device. Thus, each of the goniometers may measure the at least one selected angle.

### BACKGROUND OF THE DISCLOSURE

### Field of the Disclosure

The disclosure relates to therapy devices and more particularly pertains to a new therapy device for measuring results of a plurality of range of motion tests.

### SUMMARY OF THE DISCLOSURE

An embodiment of the disclosure meets the needs presented above by generally comprising a measuring device that may be manipulated thereby facilitating the measuring device to measure a plurality of range of motion tests. The measuring device comprises a plurality of rulers is movably coupled together. Thus, the measuring device may form at least one selected angle. A plurality of goniometers is provided. Each of the goniometers is rotatably coupled to the measuring device. Thus, each of the goniometers may measure the at least one selected angle.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective in-use view of a physical therapy assembly in a TUG position according to an embodiment of the disclosure.

FIG. 2 is a perspective in-use view of an embodiment of the disclosure in a Y balance position.

FIG. 3 is a cross sectional view taken along line 3-3 of FIG. 2 of an embodiment of the disclosure.

FIG. 4 is a perspective in-use view of an embodiment of the disclosure in a Four Square Step position.

FIG. 5 is a perspective in-use view of an embodiment of the disclosure in a Hexagon Jump Test position.

### DESCRIPTION OF THE PREFERRED EMBODIMENT

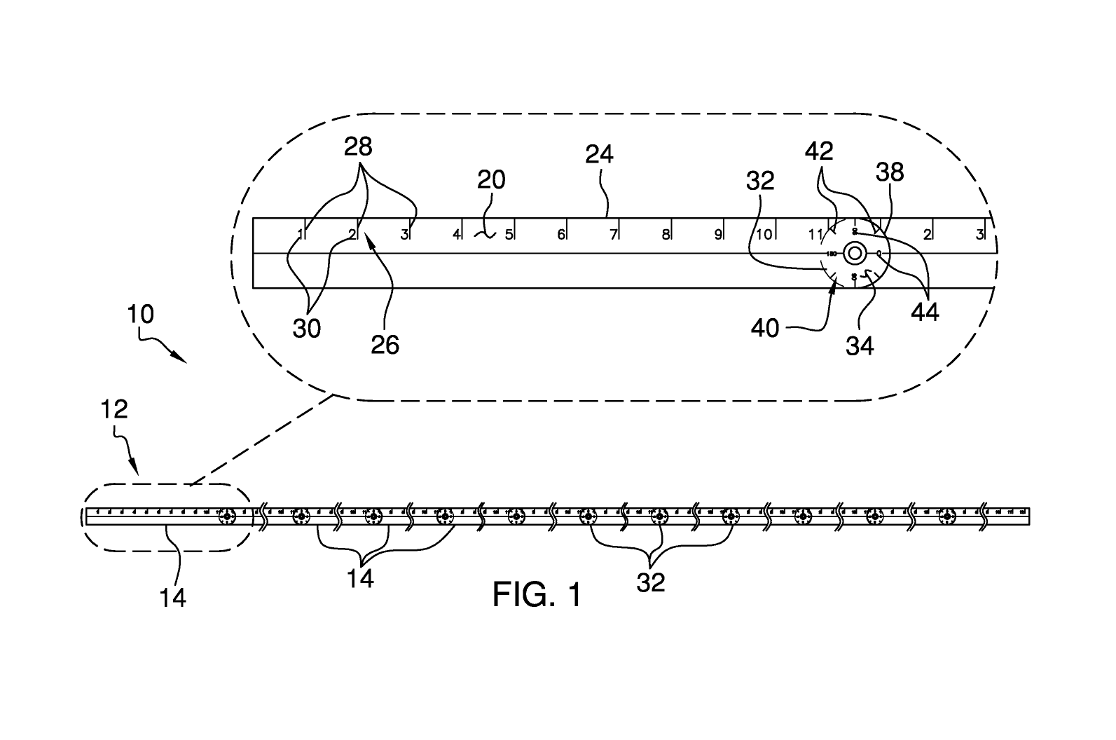

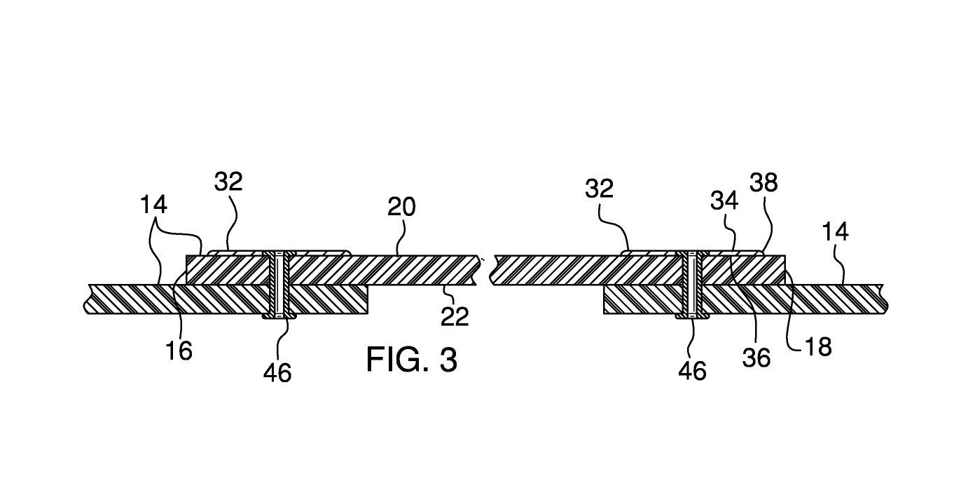

With reference now to the drawings, and in particular to FIGS. 1 through 5 thereof, a new therapy device embodying the principles and concepts of an embodiment of the disclosure and generally designated by the reference numeral 10 will be described.

As best illustrated in FIGS. 1 through 5, the physical therapy assembly 10 generally comprises a measuring device 12 that may be manipulated thereby facilitating the measuring device 12 to measure a plurality of range of motion tests. The range of motion tests include, but are not limited to, a TUG test, a Y balance Test, a Four Square Step Test and a Hexagon Jump Test. The measuring device 12 comprises a plurality of rulers 14. The rulers 14 are movably coupled together such that the measuring device 12 may form at least one selected angle.

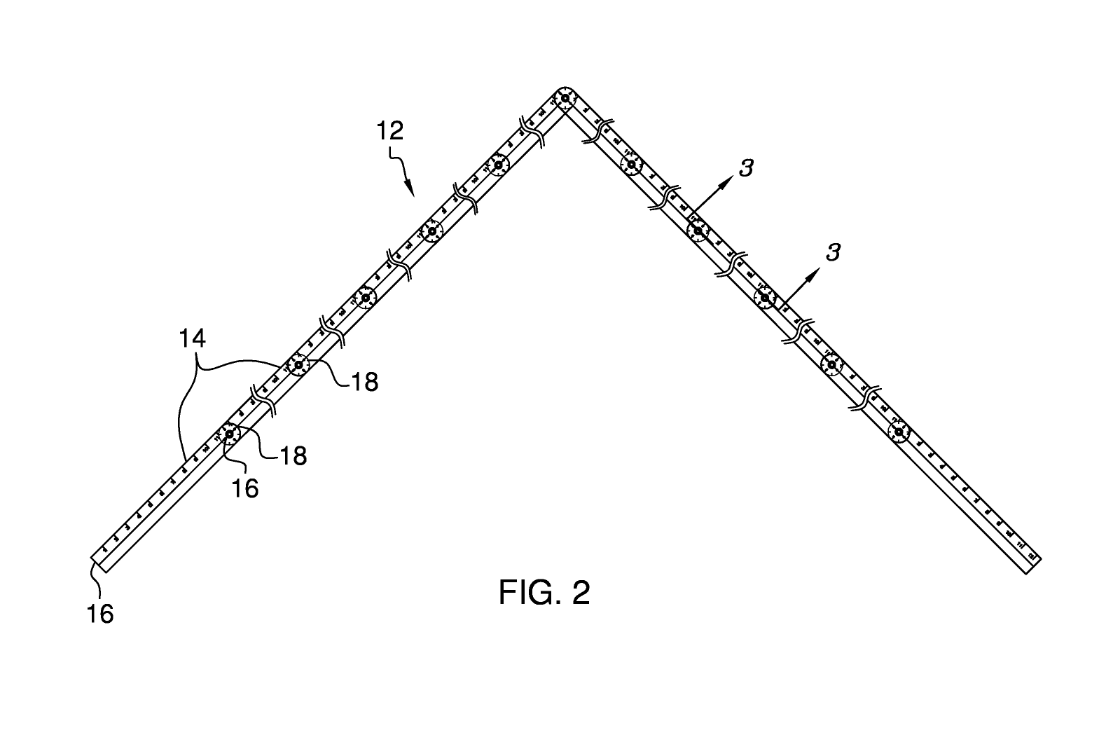

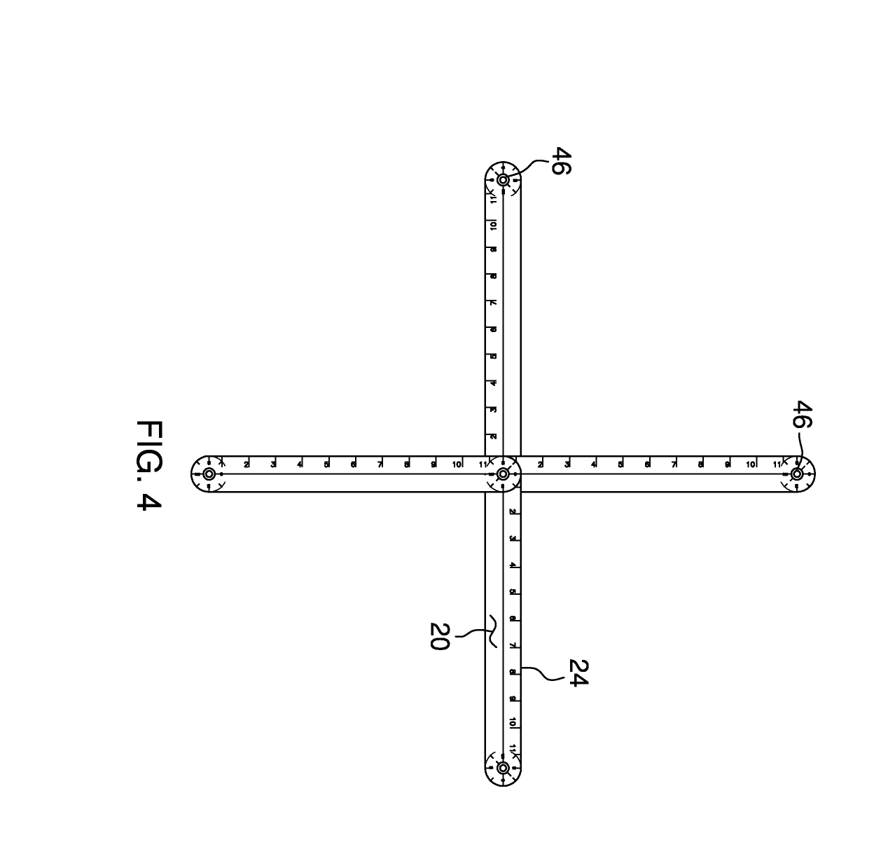

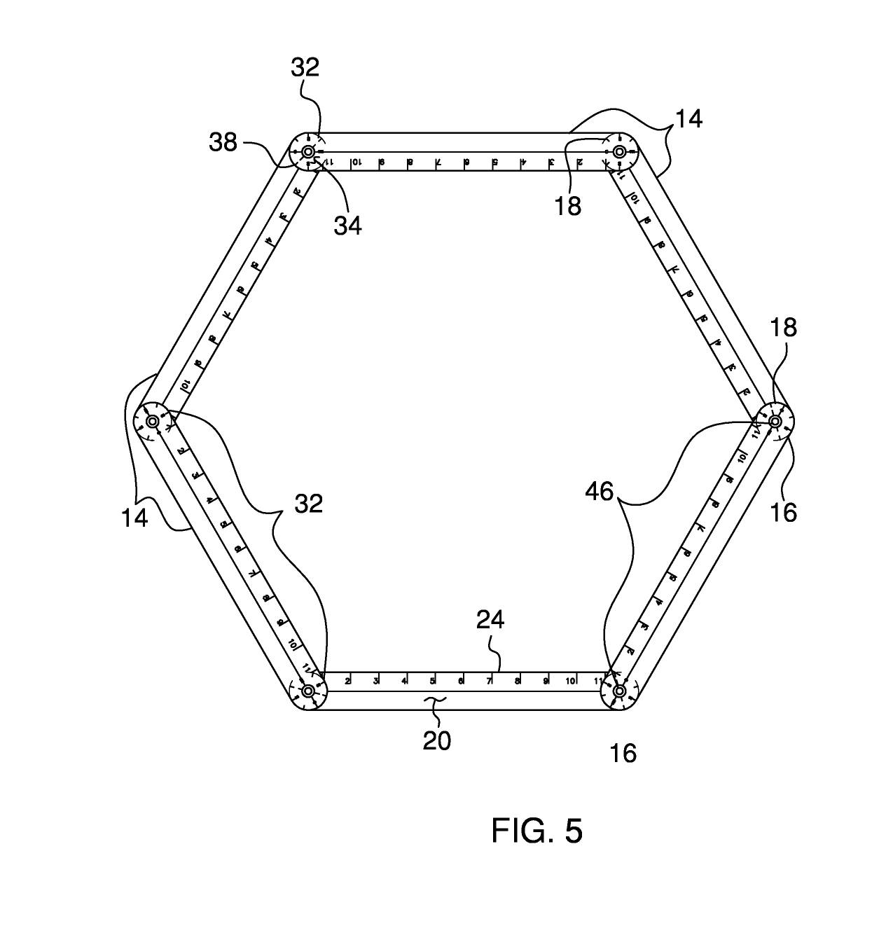

As shown in FIG. 1, the measuring device 12 is positionable in a first position such that the measuring device 12 may measure results of the TUG test. As shown in FIG. 2, the measuring device 12 is positionable in a second position such that the measuring device may measure results from a Y-Excursion Test. As shown in FIG. 4, the measuring device 12 is positionable in a third position such that the measuring device 12 may measure results of a Four Square Step Test. As shown in FIG. 5, the measuring device 12 is positionable in a fourth position such that the measuring device 12 may measure results of a Hexagon Jump Test.

Each of the rulers 14 has a first end 16, a second end 18, a top surface 20, a bottom surface 22 and a first edge 24. The top surface 20 of each of the rulers 14 has indicia 26 printed thereon. The indicia 26 comprise a plurality of lines 28 and each of the lines 28 may extend inwardly from the first edge 24. The lines 28 are spaced apart from each other and distributed between the first end 16 and the second end 18.

The indicia 26 further comprise a plurality of numbers 30 and each of the numbers 30 is aligned with an associated one of the lines 28. The numbers 30 increase in value between the first end 16 and the second end 18. Thus, each of the rulers 14 may measure distance. Each of the rulers 14 may have a length ranging between thirty cm and thirty five cm.

A plurality of goniometers 32 is provided and each of the goniometers 32 is rotatably coupled to the measuring device 12. Each of the goniometers 32 is positioned at an intersection of an associated pair of the plurality of rulers 14. Each of the goniometers 32 has a top surface 34, a bottom surface 36 and a peripheral edge 38 extending between the top surface 34 and the bottom surface 36. The peripheral edge 38 of each of the goniometers 32 is curved such that each of the goniometers 32 forms a disk.

The top surface 34 of each of the goniometers 32 has indicia 40 printed thereon. The indicia 40 corresponding to each of the goniometers 32 comprise a plurality of lines 42 extending inwardly from the peripheral edge 38. The lines 42 corresponding to each of the goniometers 32 are spaced apart from each other and are distributed around the peripheral edge 38. The indicia 40 corresponding to each of the goniometers 32 additionally comprise a plurality of numbers 44. Each of the numbers 44 corresponding to each of the goniometers 32 is aligned with an associated one of the lines 42 corresponding to each of the goniometers 32. The numbers 44 corresponding to each of the goniometers 32 corresponds to an angle ranging between zero degrees and one hundred eighty degrees. Thus, each of the goniometers 32 may indicate the at least one selected angle between the respective pair of rulers 14 when the measuring device 12 is manipulated.

A plurality of fasteners 46 is provided. Each of the fasteners 46 extends through the top surface 20 and the bottom surface 22 of the respective pair of rulers 14 such that the plurality of rulers 14 is rotatably coupled together. The bottom surface 22 of alternating ones of the rulers 14 frictionally engages the top surface 20 of alternating ones of the rulers 14. Thus, the measuring device 12 is positionable in a selected geometric shape. Each of the fasteners 46 may comprise a rivet or the like.

In use, as shown in FIG. 1, the measuring device 12 is manipulated such that the rulers 14 are laid end to end on a support surface 48 when the measuring device 12 measures to results of the TUG test. The support surface 48 may be a floor or the like. As shown in FIG. 2, the measuring device 12 is manipulated to form a right angle on the support surface 48 when the measuring device 12 measures the results of the Y balance test. As shown in FIG. 4, the measuring device 12 is manipulated to form an X on the support surface 48 when the measuring device 12 measures the results of the Four Square Step test. As shown in FIG. 5, the measuring device 12 is manipulated to form a hexagon on the support surface 48 to measure the results of the Hexagon Jump Test. The measuring device 12 is manipulated into any selected geometric shape to conduct any conceivable range of motion test.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A physical therapy assembly comprising:
a measuring device being configured to be manipulated thereby facilitating said measuring device to measure a plurality of range of motion tests, said measuring device comprising a plurality of rulers being movably coupled together wherein said measuring device is configured to form at least one selected angle; and
a plurality of goniometers, each of said goniometers being rotatably coupled to said measuring device wherein each of said goniometers is configured to measure the at least one selected angle.

2. The assembly according to claim 1, wherein:
each of said rulers has a first end, a second end, a top surface, a bottom surface and a first edge, said top surface of each of said rulers having indicia being printed thereon; and
said indicia comprising
a plurality of lines, each of said lines extending inwardly from said first edge, said lines being spaced apart from each other and distributed between said first end and said second end, and
a plurality of numbers, each of said numbers being aligned with an associated one of said lines, said numbers increasing in value between said first end and said second end wherein each of said rulers is configured to measure distance.

3. The assembly according to claim 2, wherein said measuring device is positionable in a first position wherein said measuring device is configured to measure results of a TUG test.

4. The assembly according to claim 2, wherein said measuring device is positionable in a second position wherein said measuring device is configured to measure results from a Y balance test.

5. The assembly according to claim 2, wherein said measuring device is positionable in a third position wherein said measuring device is configured to measure results of a Four Step Square test.

6. The assembly according to claim 2, wherein said measuring device is positionable in a fourth position wherein said measuring device is configured to measure results of a Hexagon Jump test.

7. The assembly according to claim 1, wherein each of said goniometers is positioned at an intersection of an associated pair of said plurality of rulers such that each of said goniometers measures a selected angle between said respective pair of rulers when said measuring device is manipulated, each of said goniometers having a top surface, a bottom surface and a peripheral edge extending between said top surface and said bottom surface, said peripheral edge of each of said goniometers being curved such that each of said goniometers forms a disk.

8. The assembly according to claim 7, wherein said top surface of each of said goniometers has indicia being printed thereon, said indicia comprising:
a plurality of lines extending inwardly from said peripheral edge, said lines corresponding to each of said goniometers being spaced apart from each other and being distributed around said peripheral edge; and
a plurality of numbers, each of said numbers corresponding to each of said goniometers being aligned with an associated one of said lines corresponding to each of said goniometers such that each of said goniometers is configured to measure the at least one selected angle between said respective pair of rulers.

9. The assembly according to claim 1, further comprising:
each of said plurality of rulers having a top surface and a bottom surface; and
a plurality of fasteners, each of said fasteners extending through said top surface and said bottom surface of said respective pair of rulers and an associated one of said goniometers such that said plurality of rulers are rotatably coupled together, said bottom surface of alternating ones of said rulers frictionally engaging said top surface of alternating ones of said rulers.

10. A physical therapy assembly comprising:
a measuring device being configured to be manipulated thereby facilitating said measuring device to measure a plurality of range of motion tests, said measuring device comprising a plurality of rulers, said rulers being movably coupled together wherein said measuring device is configured to form at least one selected angle, said measuring device being positionable in a first position wherein said measuring device is configured to measure results of a TUG test, said measuring device being positionable in a second position wherein said measuring device is configured to measure results from a Y-Excursion test, said measuring device being positionable in a third position wherein said measuring device is configured to measure results of a Four Square Step test, said measuring device being positionable in a fourth position wherein said measuring device is configured to measure results of a Hexagon Jump test, each of said rulers having a first end, a second end, a top surface, a bottom surface and a first edge, said top surface of each of said rulers having indicia being printed thereon, said indicia comprising:
a plurality of lines, each of said lines extending inwardly from said first edge, said lines being spaced apart from each other and distributed between said first end and said second end, and
a plurality of numbers, each of said numbers being aligned with an associated one of said lines, said numbers increasing in value between said first end and said second end wherein each of said rulers is configured to measure distance;
a plurality of goniometers, each of said goniometers being rotatably coupled to said measuring device, each of said goniometers being positioned at an intersection of an associated pair of said plurality of rulers such that each of said goniometers measures the at least one selected angle between said respective pair of rulers when said measuring device is manipulated, each of said goniometers having a top surface, a bottom surface and a peripheral edge extending between said top surface and said bottom surface, said peripheral edge of each of said goniometers being curved such that each of said goniometers forms a disk, said top surface of each of said goniometers having indicia being printed thereon, said indicia comprising:
a plurality of lines extending inwardly from said peripheral edge, said lines corresponding to each of said goniometers being spaced apart from each other and being distributed around said peripheral edge, and
a plurality of numbers, each of said numbers corresponding to each of said goniometers being aligned with an associated one of said lines corresponding to each of said goniometers such that each of said goniometers is configured to indicate the at least one selected angle between said respective pair of rulers; and
a plurality of fasteners, each of said fasteners extending through said top surface and said bottom surface of said respective pair of rulers such that said plurality of rulers are rotatably coupled together, said bottom surface of alternating ones of said rulers frictionally engaging said top surface of alternating ones of said rulers.
